(19) United States
(12) Patent Application Publication
(10) Pub. No.: US 2004/0054392 A1
(43) Pub. Date: Mar. 18, 2004

(54) Probe for treatment of incotinence

(76) Inventor: Gerrat Dijkman, Enschede (NL)

Correspondence Address: JENSEN + PUNTIGAM, P.S., SUITE 1020, 2033 6TH AVE, SEATTLE, WA, 98121, US

(21) Appl. No.: 10/381,388

(22) PCT Filed: Sep. 20, 2001
(86) PCT No.: PCT/NL01/00696

(30) Foreign Application Priority Data
Sep. 21, 2000 (NL) 1016228

(51) Int. Cl. A61N 1/05

(52) U.S. Cl. 607/138

(57) ABSTRACT

Probe for treatment of urinary or faecal incontinence, which probe is substantially intended for intra-vaginal respectively intra-anal use and is provided on an outer surface with at least two electrodes. The probe comprises a substantially hollow body of a flexible material, and is provided with means for measuring pressure in the body, EMG and performing electro-stimulation.

The invention relates to a probe for treatment of urinary or faecal incontinence, which probe is substantially intended for intra-vaginal respectively intra-anal use and is provided on an outer surface with it least two electrodes.

An intra-vaginal or intra-anal probe provided with electrodes is generally known in the treatment of incontinence, both for diagnostic and therapeutic purposes.

As diagnostic aid, the known probe is introduced intra-vaginally or intra-anally and the electric voltage is measured over the at least two electrodes. Such a measurement is known as electromyography (EMG). The result of a measurement with EMG gives an indication of the electrical activity of the sphincter vesicae (contractor of the bladder) respectively the sphincter anl (contractor of the anus) and of the pelvic floor as a whole. As therapeutic aid the known probe is applied to prickle and thus stimulate contraction of the muscle in question by applying a voltage over the at least two electrodes. In order to be able to assess whether a determined EMG activity also results in the desired contraction, and therefore whether electro-stimulation is a useful therapy, it must first be determined whether the relevant muscle is still able to contract. This final determination is carried out using an intra-vaginal or intra-anal pressure probe, or by means of a vaginal or anal internal examination, the outcome of which is frequently unclear.

Working with different probes for diagnostic and therapeutic purposes has the effect of increasing costs while the risk of infections is moreover increased.

It is an object of the invention to provide a probe which is suitable for both therapeutic and diagnostic purposes in the field of electro-stimulation, and which is also suitable for performing pressure measurements.

This object is achieved, and other advantages gained, with a probe of the type specified in the preamble, wherein according to the invention the probe comprises a substantially hollow body of a flexible material, and is provided with means for measuring pressure in the body.

In a probe according to the invention the flexible walls of the hollow body act as transfer medium to transfer the pressure on the probe onto the interior of the probe, which pressure can be determined with the pressure measuring means.

In an embodiment of a probe, the hollow body is provided with a sealable opening, so that a bias can be adjusted in simple manner in the interior of the body.

In a practical embodiment the opening in the body can be sealed by a non-return valve.

The body is for instance manufactured from silicone material or from polyurethane (PUR). Both materials are sufficiently flexible for the intended application and suitable for medical use.

In a further embodiment the electrodes extend in longitudinal direction over the outer surface of the body. The measuring surface is hereby enlarged and the accuracy of positioning increased in advantageous manner. Application of elongate electrodes further enables the use of a larger number of electrodes than the two that are usual in practice, which provides additional options such as locally selective measurement and/or stimulation.

The probe is preferably provided with at least three electrodes, one of which serves as earth connection, so that the probe can be used to perform an EMG measurement without applying additional electrodes.

In a particularly advantageous embodiment the electrodes comprise electrically conducting silicone material.

In preference the body is generally cylinder-shaped, which fits well in the bodily orifice(s) for which the probe is intended,

The body of the probe according to the invention is advantageously obtained by extrusion,

The invention will be elucidated hereinbelow on the basis of an embodiment and with reference to the drawing, in which

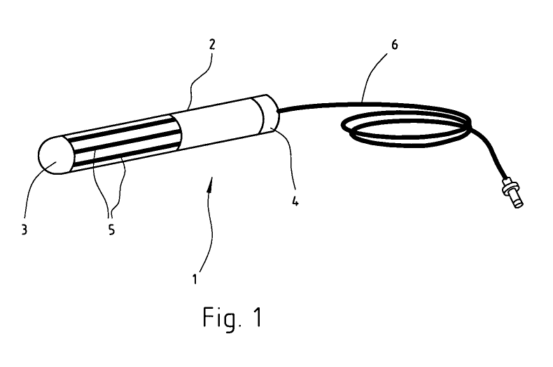

FIG. 1 shows a schematic view of a probe according to the invention; and

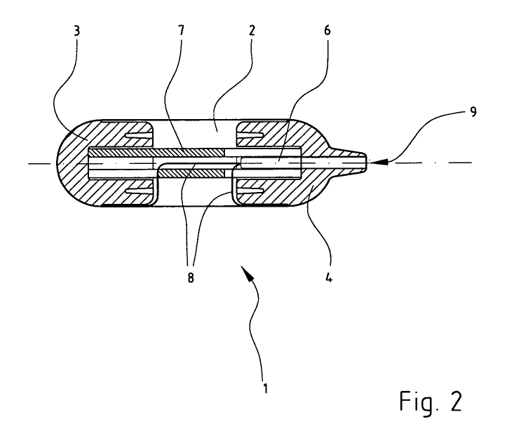

FIG. 2 shows the probe according to FIG. 1 in longitudinal section.

In the drawing FIGS. 1 and 2 show a probe 1 intended for vaginal use. Probe 1 comprises a hollow body 2 which in this embodiment has a general cylinder shape and is manufactured from a flexible material suitable for medical application, for instance silicone material. An example of a suitable material is also designated. in the field as silicone rubber. Cylinder 2 extends between a front end cap 3 and a rear end cap 4 of a suitable solid plastic material.

Electrodes 5 of an electrically conducting material suitable for medical application extend in longitudinal direction over the outer surface of cylinder 2. The above stated silicone material or silicone rubber is very suitable for this purpose. Electrodes 5 are connected to the cores 8 of a cable 6 guided through the rear end cap 4. It is noted that the probe according to the invention is suitable for connection to all electrical equipment known in the technical field for the purpose of diagnosis and treatment of incontinence, particularly by means of electro-stimulation.

Cylinder 2 is sealable by means of a non-retum valve (not shown) to bring the interior of cylinder 2 to a desired bias. Arranged for this purpose in cable 6 is an air channel (not shown) which is connected to the interior of cylinder 2. The non-return valve serves to seal this air channel. Alternatively, the non-return valve can be arranged directly in opening 9 in rear end cap 4. No use is then made of cable 6.

A pressure sensor (not shown) is also provided for determining the pressure in the interior of cylinder 2. For this purpose the interior of cylinder 2 is placed in pressure connection, via the air channel in cable 6, with a separate apparatus (not shown) for measuring the pressure, wherein the pressure sensor is received in the apparatus. In the above stated alterative, in the absence of cable 6, the pressure sensor can be arranged in probe 1, for instance in rear end cap 4.

For strength ning purposes th end caps are mutually connected by a plastic rod 7, which also prevents undesired contact between electrod s 5. Rod 7 preferably takes a hollow form, so that electrical connecting wires 8 of electrodes 5 can be carried therein.

In practice electro-stimulation and measuring of EMG activity take place in the usual manner as generally applied in the field with known probes. The elongate electrodes 5 herein provide a number of significant advantages. Firstly, they provide a large measuring surface so that the relevant muscle can be measured/stimulated over a large part of the surface thereof. Owing to the large electrode surface, the probe can moreover be positioned correctly in the body of the patient by means of a fast and simple operation. In addition, the number of electrodes can be increased as desired, to for instance six, which provides the option of selective, local measurement/stimulation.

During a pressure measurement the body of the probe must be sealed. If desired, the probe can first be brought to an increased bias. The material of the hollow body 2 is preferably flexible for this purpose such that the diameter of hollow body 2 can be inflated to 1.5 times or even twice the diameter of the end caps. This has the advantage that the dimension of the probe can be adjusted to the dimension of the patient, which increases the effectiveness of the measurement/stimulation. The pressure which the patient exerts on the wall of hollow body 2 by contracting the relevant muscle results in a pressure increase in the interior of hollow body 2 which is measured by the pressure sensor.

The flexibility and resilience which are inherent to the probe according to the invention are maintained during use. This has the significant advantage in the treatment of incontinence that the muscle formation of the muscle under treatment in this case is additionally stimulated by the resistance encountered by this muscle during the exertion of pressure on the probe, which increases the effectiveness of the treatment.

It is finally noted that the probe according to the invention can be manufactured at a low cost price using the above stated materials. This makes the probe highly suitable for application as disposable article, i.e. the probe is then preferably intended for (re)use by one patient.

It is noted that in principle there is no difference between an intra-vaginal and an intra-anal probe, other than in shape and dimensions.

1. Probe (1) for treatment of urinary or faecal incontinence, which probe is substantially intended for intra-vaginal respectively intra-anal use and is provided on an outer surface with at least two electrodes (5), characterized in that the probe (1) comprises a substantially hollow body (2) of a flexible material, and is provided with means for measuring pressure in the body (2).

2. Probe as claimed in claim 1, characterized in that the body (2) is provided with a sealable opening.

3. Probe as claimed in claim 2, characterized in that the opening can be sealed by a non-return valve.

4. Probe as claimed in any of the claims 1-3, characterized in that the body (2) is manufactured from silicone material.

5. Probe as claimed in any of the claims 1-4, characterized in that the electrodes (5) extend in longitudinal direction over the outer surface of the body.

6. Probe as claimed in any of the claims 1-6, characterized in that the probe (1) is provided with at least three electrodes.

7. Probe as claimed in any of the claims 16, characterized in that the electrodes (5) comprise electrically conducting silicone material.

8. Probe as claimed in any of the claims 1-7, characterized in that the body (2) is manufactured from polyurethane (PUR).

9. Probe as claimed in any of the claims 1-8, characterized in that the body (2) is obtained by extrusion.

10. Probe as claimed in any of the claims 1-9, characterized in that the body (2) has a generally cylindrical shape.
